(19) United States
(12) Patent Application Publication
(10) Pub. No.: US 2022/0211726 A1
(43) Pub. Date: Jul. 7, 2022

(54) UVEAL MELANOMA TREATMENT

(71) Applicant: Katholieke Universiteit Leuven, Leuven (BE)

(72) Inventors: Eleonora LEUCCI, Linden (BE); Roberto VENDRAMIN, Brussel (BE)

(21) Appl. No.: 17/601,320

(22) PCT Filed: Apr. 2, 2020
(86) PCT No.: PCT/EP2020/059416

§ 371 Date: Oct. 4, 2021

(30) Foreign Application Priority Data
Apr. 5, 2019 (GB) 1904851.1

(51) Int. Cl. A61K 31/65; A61K 31/7036; A61K 45/06; A61P 35/04

(52) U.S. Cl. CPC A61K 31/65; A61P 35/04; A61K 45/06; A61K 31/7036

(57) ABSTRACT

The invention relates to antibiotics inhibiting the 30S ribosomal subunit, such as tigecycline, for use in the treatment of uveal melanoma.

### FIELD OF THE INVENTION

The invention relates to the use of antibiotics in cancer therapy.

The invention further relates to treatments of specific types of eye melanoma.

### BACKGROUND OF THE INVENTION

Antibiotics have been developed and used for the treatment of various bacterial infections. Recently, antibiotics have been used as well in the field of cancer therapy. Tigecycline is an antibiotic of the glycycyclin class developed for the treatment of antibiotic resistant bacteria.

Hu et al. (2016) Oncotarget 7(3), 3171-3185 discloses the use of tigecycline on A375 and MV3 cutaneous melanoma cell lines, which is distinct medical entity from uveal melanoma.

Kaliki & Shields (2017) Eye (Lond) 31(2), 241-257 refer to uveal melanoma as a rare but deadly cancer.

Lamb et al (2015) Oncotarget. 6, 4569-458 describe the use of tigecycline on A375 skin melanoma cell line.

A recent review on uveal melanoma by Yang et al. (2018) Ther Adv Med Oncol. 10: 1758834018757175, mentions that nearly 50% of patients will develop metastatic disease. The outcome for those with metastatic disease remain dismal due to a lack of effective therapies.

There is a need for further therapies for uveal melanoma.

### SUMMARY OF THE INVENTION

The present invention shows that exposure of a uveal melanoma cell line to increasing concentrations of the antibiotic Tigecycline leads to a significant and dose-dependent decrease in cell growth.

The present invention illustrates that targeting mitochondrial translation with mitoribosome-targeting antibiotics alone, significantly slowed down progression of uveal melanoma both in vitro and in preclinical settings. Similar effects were observed using in vitro and in vivo uveal melanoma models. This observation offers a new avenue for the treatment of these diseases considering that, to date, only a limited number of therapeutic options exist.

The invention is summarized in the following statements:

1. An antibiotic inhibiting the 30S ribosomal subunit, for use in the treatment of uveal melanoma.

2. The antibiotic for use in according to claim 1, wherein the antibiotic is an aminoglycoside or tetracycline.

3. The antibiotic for use in the treatment according to claim 1 or 2, wherein the tetracycline is a glycylcycline.

4. The antibiotic for use in the treatment according to claim 3, wherein the glycylcycline is tigecyline.

5. The antibiotic t for use in the treatment according to any one of claims 1 to 4, wherein the uveal melanoma is in metastasis.

6. The antibiotic for use in the treatment according to any one of claims 1 to 5, wherein the uveal melanoma carries a mutation in GNAQ, GNA11, BAP1, SF3B1, EIFAX1 or TERT.

7. The antibiotic for use in the treatment o according to any one of claims 1 to 6, wherein the antibiotic is administered intravenously, per OS or intra.

8. The antibiotic for use in the treatment according to any one of claims 1 to 7, wherein the treatment is in combination with another chemotherapy.

9. The antibiotic for use in the treatment according to any one of claims 1 to 8, wherein the treatment is performed in the absence a of radiochemical.

10. A method of treating a uveal melanoma comprising the step of administering of an antibiotic inhibiting the 30S ribosomal subunit

11. The method according to claim 10, wherein the antibiotic is an aminoglycoside or a tetracycline.

12. The method according to claim 11, wherein the tetracycline is a glycylcycline.

13. The method according to claim 12, the glycylcycline is tigecyline.

### DETAILED DESCRIPTION

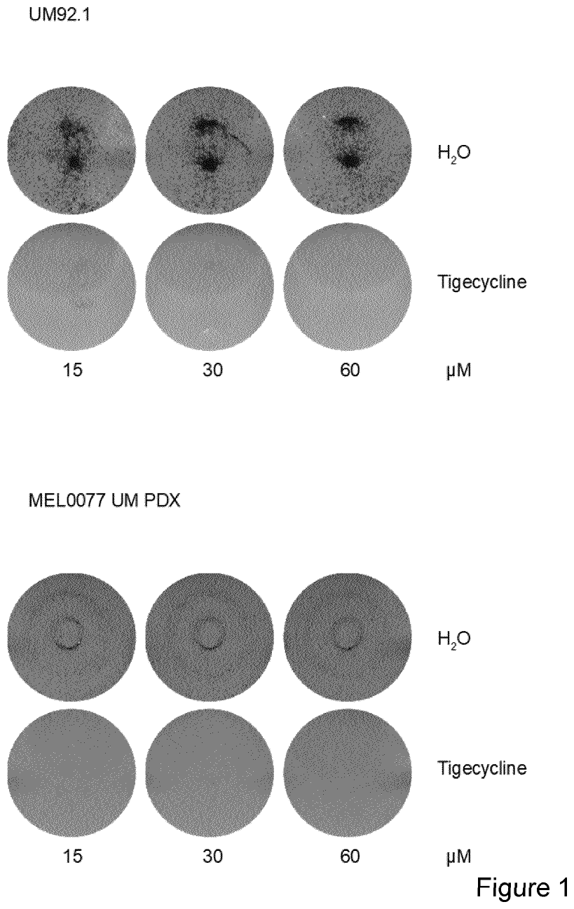

FIG. 1 shows the in vitro effect of tigecycline on two different uveal melanoma cell lines.

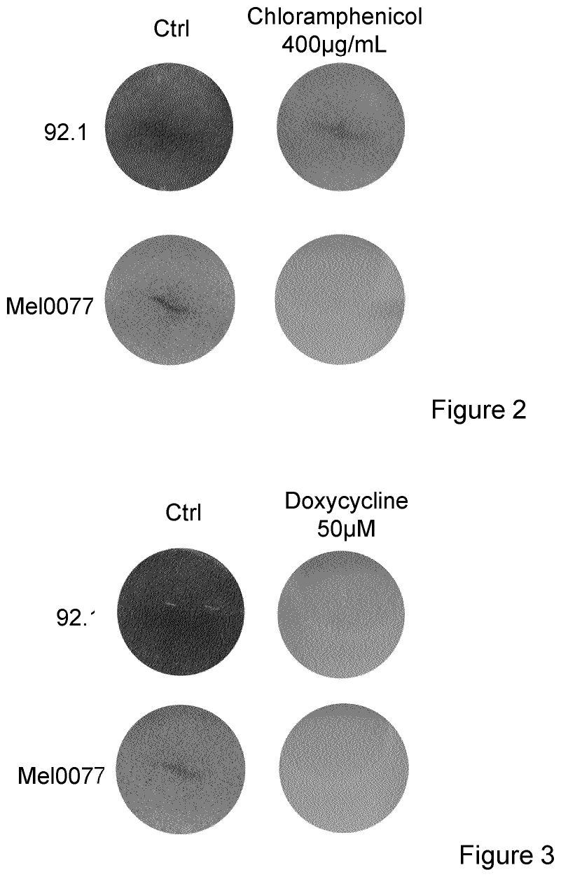

FIG. 2 shows the in vitro effect of chloramphenicol on two different uveal melanoma cell lines.

FIG. 3 shows the in vitro effect of doxycycline on two different uveal melanoma cell lines.

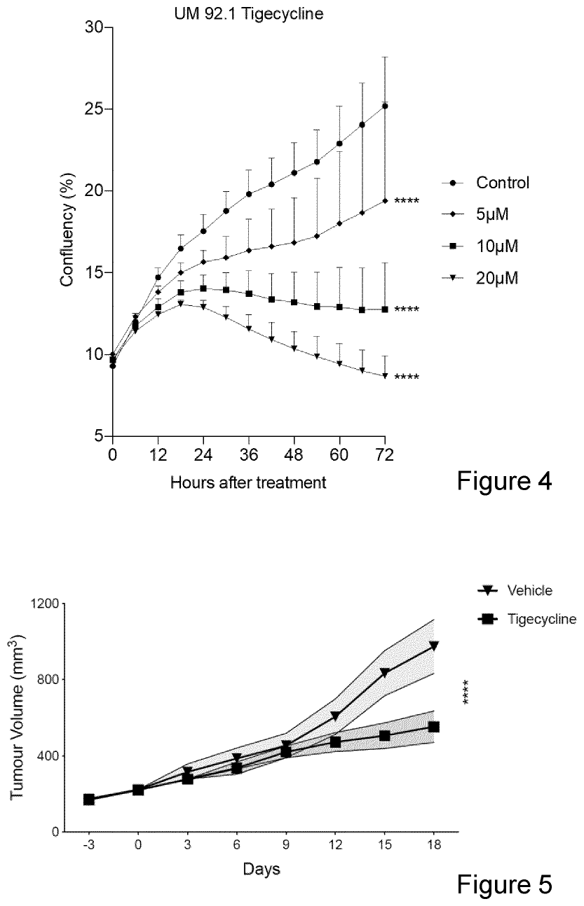

FIG. 4 shows the effect of tigecycline on a GNAQ mutant uveal melanoma (UM 92.1) cell growth (measured as % of cell confluency) upon exposure to increasing concentrations of Tigecycline for 72 h. Data are the means±s.e.m. of three independent experiments. P values were calculated by paired t-test.

FIG. 5 shows that melanoma lesions intrinsically resistant to immunotherapy are sensitive to Tigecycline. The figure shows tumour volume of cohorts of Mel-077 uveal melanoma PDX mice treated with vehicle (DMSO) or Tigecycline. Data are the means s.e.m of different biological replicates. P value was calculated by two-ways ANOVA.

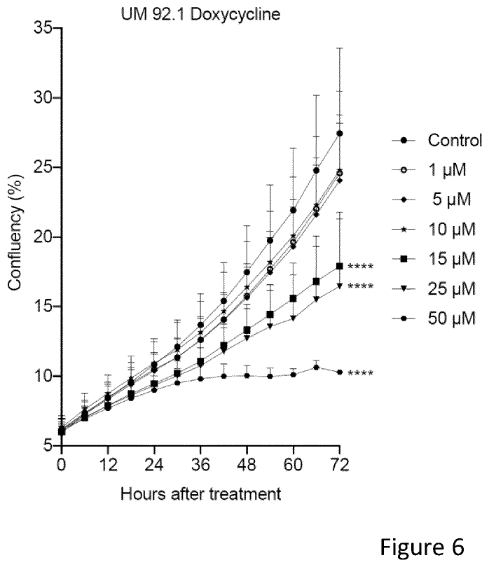

FIG. 6 shows effect of Doxycycline on uveal melanomas Cell growth (measured as % of cell confluency) of a uveal melanoma (UM 92.1) cell line of cells described in upon exposure to increasing concentrations of Doxycycline Data are the means±s.e.m. of three independent experiments. P values were calculated by paired t-test.

### DEFINITIONS

“Uveal melanoma” refers to a melanoma of the choroid, ciliary body, and iris of the eye.

Therapy of uveal melanoma has been performed with laser therapy, radiation therapy and removal of the eye globe.

A clear distinction is to be made from certain type of cutaneous melanoma. In this context Chattopandyay et al. (2016) Cancer. 122(15), 2299-2312 mention that the terms choroidal or ocular melanoma are used as alternative terms for this cancer as the majority of the uveal tract is choroidal, although the latter term should be avoided as it implies the inclusion of conjunctival and adnexal melanomas, which behave and are managed like cutaneous rather than uveal primaries.

The clinical behaviour of uveal melanomas can be segregated into two main groups: a) those that are diagnosed and confined locally to the eye, and b) those that metastasize and are ultimately fatal from distant disease. Uveal melanomas with chromosome 3 loss confer the worst prognosis, while those with 6p gain have the best outcomes.

Gene expression profiling using an RNA-based assay demonstrates clustering into Class 1 tumours, those with low metastatic potential, and Class 2 tumors, those with high metastatic potential. This test, is marketed as DecisionDx-UM® (Castle Biosciences, Friendswood, Tex.) and is described in Harbour (2014) Methods Mol Biol. 1102, 427-440.

Another uveal melanoma prognostic test from Impact Genetics (Ontario, Canada) assays copy numbers of chromosomes 1p, 3, 6, and 8, along with microsatellite analysis of chromosome 3 and confirmation of GNAQ or GNA11 gene mutation status [Damato & Coupland (2009) Arch Ophthalmol. 127, 423-429].

Oncogene and tumor suppressor mutations that are common in other cancers are mostly absent in uveal melanoma, a disease characterized by low mutation burden. It also differs in genetic mutation profile from conventional cutaneous melanoma where BRAF and NRAS mutations dominate. These “driver” mutations that control the biology of up to 70% of cutaneous melanomas are absent/rare in uveal melanoma. Primary uveal melanoma obtained from enucleation reveal mutations in the genes GNAQ, GNA11, BAP1, SF3B1, EIFAX1 and TERT.

GNAQ/GNA11 [guanine nucleotide-binding protein G(q) subunit alpha (Gαq)/alpha subunit of G11 G protein]. The majority of uveal melanomas have one of two mutually exclusive activating mutations in the very homologous genes encoding Ga subunits, GNAQ (Gαq) and GNA11 (Gα11) (19-21). The GNAQ mutation is more frequently found in benign blue nevi, while the GNA11 mutation is frequent in malignant uveal melanoma.

BAP1: somatic mutations have been described in the BAP1 gene (BRCA1-associated protein 1), on chromosome 3p21.1

SF3B1: SF3B1 gene encodes the splicing factor 3B subunit 1. Uveal melanoma is among a small group of cancers associated with SF3B1 mutations. These mutations define a genetic subset of uveal melanoma to be associated with favorable prognostic features and to be nearly always mutually exclusive of BAP1 mutations.

EIF1AX: recurrent somatic mutations in EIF1AX [Eukaryotic Translation Initiation Factor 1A X-Linked] have been detected along with SF3B1, specifically occurring in uveal melanomas with disomy 3, which rarely metastasize. EIF1AX mutations are infrequent in monosomy 3 uveal melanomas.

Van der Kooij et al. (2019) Cancers 11 845 review the difference between cutaneous melanoma (CM) and uveal melanoma (UV).

Cutaneous melanoma and conjunctival melanoma are genetically distinct from uveal melanoma. The majority of cutaneous melanoma cases harbour mutations in proteins associated with the mitogen-activated protein kinase (MAPK) pathway. BRAF kinase mutations are present in 40-60% of the cutaneous melanoma patients, 97% of which is located in codon 600.

The second most common MAPK pathway aberration in cutaneous melanoma is mutated NRAS, occurring in 15-30% of patients Melanoma with mutations in the stem cell factor receptor tyrosine kinase gene (KIT) represents a relatively rare subset, seen in roughly 20% of mucosal, acral, and chronically sun-damaged skin. In uveal melanoma, the most commonly mutated genes are GNA11, GNAQ, BAP1, EIF1AX, and SF3B1.

Primary uveal melanoma can be stratified into four distinct, clinically relevant molecular subtypes with a significant difference in metastatic rate and prognosis Class 1A and 1B tumours retain a differentiated melanocyte phenotype, with a disomy of chromosome 3. They are further distinguished by alterations in either EIF1AX or SF3B1, respectively, with 1A having a lower metastatic rate when compared to 1B. Class 2 uveal melanoma is associated with a high metastatic risk and is characterized by a monosomy of chromosome 3, followed by aberrancies in BAP1 expression and global DNA methylation. A further subdivision can be made into class 2A and 2B based on chromosome 8q copy number alterations, RNA expression, and cellular pathway activity profiles, with Class 2B having a higher metastatic rate when compared to Class 2A.

The difference between cutaneous and uveal melanoma at the molecular level is also reflected in the responsiveness for therapeutic agents. A therapeutic effect of a medicament against cutaneous melanoma does not imply or suggest an effect against uveal melanoma.

Indeed, Pandiani et al. (2017) Genes & Dev. 31, 724-743 disclose in the abstract of the article that therapeutic improvements achieved in the last few years for the treatment of cutaneous melanoma have failed to ameliorate the clinical outcomes of patients with uveal melanoma.

Equally, Carvajal et al. (2017) Br J Ophthalmol. 101, 38-44 cite that Dacarbazine, a chemotherapeutic option for treatment of cutaneous melanoma, has been used for uveal melanoma, despite the inherent differences between these molecularly distinct diseases and activity has been limited. The authors further mention that other chemotherapeutic regimens including temozolomide, cisplatin, treosulfan, fotemustine and various combinations have been investigated in uveal melanoma with disappointing results to date.

The present invention relates to the use of certain antibiotics for treating a type or class of uveal melanoma as described above.

Suitable antibiotics in the context of the present invention are antibiotics inhibiting 30S ribosomal subunit inhibitors.

Antibiotics that affect the ribosome are reviewed e.g. in Lambert (2012) Rev. sci. tech. Off. int. Epiz. 31, 57-64.

This class of 30S ribosomal subunit inhibitors includes “aminoglycosides”.

Aminoglycosides inhibit bacterial protein synthesis by pleiotropic actions that lead to the alteration of translation at diverse steps including initiation, elongation and termination. Aminoglycosides target the 16S ribosomal (r)RNA and particularly the decoding A-site for the 4,6-substituted 2-deoxystreptamine (2-DOS). This binding stabilises a normal mismatch in codon-anticodon pairing, leading to mistranslations. Adenine 1408 of 16S RNA is crucial for binding.

Examples of aminoglycosides include amikacin, gentamicin and tobramycin, typically used in human clinical settings, and apramycin and fortimicin typically used in veterinary medicine. Other examples are Kanamycin, Streptomycin, Neo-Fradin and Neomycinn Dibekacin, Sisomicin, Netilmicin, Neomycins B, C, E.

4,6-disubstituted and 4,5-disubstituted sub-classes of aminoglycosides are known.

In the art, aminoglycosides are typically administered intraveneously or intramuscularly.

Tetracyclines are closely related structurally, with a fourring carbocyclic skeleton that results from the biosynthesis of polyketides by bacterial type II polyketide synthases produced by Streptomyces. Tetracyclines inhibit protein synthesis by impairing the stable binding of aminoacyl-transfer (t)RNA to the bacterial ribosomal A-site. Examples of naturally occurring tetracyclines are tetracycline, chlortetracycline, oxytetracycline and emeclocycline. Examples of semi-synthetic tetracyclines are lymecycline, meclocycline, methacycline, minocycline, rolitetracycline and Doxycycline.

A further class of compounds which belong to the class of tetracylines are glycycycline.

Tigecycline is an example of glycylcycline. Tigecycline is a minocycline derivative with a substitution that increases the spectrum of activity. The structure of tigecycline is shown below:

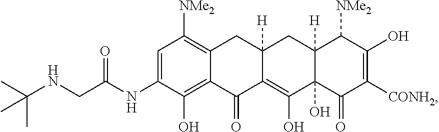 [SMILES: CN(C)c1cc(NC(=O)CNC(C)(C)C)c(O)c2c1C[C@H]1C[C@H]3[C@H](N(C)C)C(O)=C(C(N)=O)C(=O)[C@@]3(O)C(O)=C1C2=O]

Tigecyline is a 7-dimethylamino-tetracycline having an N-alkylglycylamido side chain at position 9 of the four-ring core. EP2568987 discloses other 7-dimethylamino-tetracyclines or tautomers thereof with general structure, which are suitable in the context of the present invention:

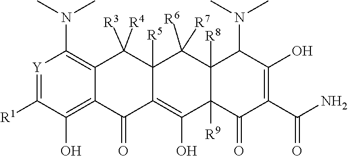

Wherein

R¹ is selected from H, —(CH₂)ₙNHC(O)(CH₂)ₙR¹⁰, and —(CH₂)ₙR¹⁰, where each n is independently an integer from 0 to 3, and

R¹⁰ is selected from —NH—C₁₋₈alkyl, —NH—C₁₋₈cycloalkyl, and a saturated 4-to-7-membered heterocycle containing one nitrogen atom, wherein if the connecting atom of R¹⁰ is carbon, the nitrogen atom is optionally substituted by C₁-C₄ alkyl;

Y is CR² or N; and

R², R³, R⁴, R⁵, R⁶, R⁷, R⁷, R⁸, and R⁹ are each independently selected from H, —OH, halogen, and C₁-4 alkyl; or optionally R¹ and R² together form a 6-membered aryl or heteroaryl ring, optionally substituted by one or two groups independently selected from H, R¹, —OH, halogen, and C₁₋₄ alkyl.

In some embodiments, each of R³, R⁴, R⁵, R⁶, R⁷, R⁸, and R⁹ are hydrogen.

“pharmaceutical composition” is defined herein to refer to a mixture or solution containing at least one therapeutic agent to be administered to a subject, e.g., a mammal or human, in order to prevent or treat a particular disease or condition affecting the mammal or human.

“pharmaceutically acceptable” is defined herein to refer to those compounds, materials, compositions and/or dosage forms, which are, within the scope of sound medical judgment, suitable for contact with the tissues a subject, e.g., a mammal or human, without excessive toxicity, irritation allergic response and other problem complications commensurate with a reasonable benefit/risk ratio.

The term “treating” or “treatment” as used herein comprises a treatment relieving, reducing or alleviating at least one symptom in a subject or effecting a delay of progression of a disease. For example, treatment can be the diminishment of one or several symptoms of a disorder or complete eradication of a disorder, such as cancer. Within the meaning of the present invention, the term “treat” also denotes to arrest, delay the onset (i.e., the period prior to clinical manifestation of a disease) and/or reduce the risk of developing or worsening a disease. The term “protect” is used herein to mean prevent delay or treat, or all, as appropriate, development or continuance or aggravation of a disease in a subject, e.g., a mammal or human.

The term “pharmaceutically effective amount” or “clinically effective amount” of a combination of therapeutic agents is an amount sufficient to provide an observable improvement over the baseline clinically observable signs and symptoms of the disorder treated with the combination.

The present invention illustrates that a daily monotreatment with Tigecycline (50 mg/kg) of the uveal melanoma PDX model Mel-077, derived from a patient progressing on the immune checkpoint inhibitor pembrolizumab (an anti-PD1 agent), was sufficient to stabilise tumour growth and significantly delay progression. These data illustrate that exposure to an antibiotic of the tetracycline family, such as tigecycline is sufficient to affect the growth of immunotherapy resistant skin and uveal melanomas even when used as a single agent.

### EXAMPLES

### Example 1. Methodology

### Methods

### Cell Viability Assays

For colony formation assays, cells were plated in six-well plates at the appropriate density (1.5×10⁴ to 8×10⁴). Cells were then treated with increasing amounts of Tigecycline and cultured for 3 days, 5 days or 7 days. Cells were then washed twice with PBS, fixed and stained for 15 min with a 1% crystal violet in 35% methanol solution.

For IncuCyte Proliferation Assays, cells were plated in 96-well plates (TPP) at the appropriate density (between 2.5×10³, to 1.5×10⁴). Cells were treated with increasing amounts of Tigecycline or Doxycycline and cultured for 72 h. Apoptotic cells were labelled with the IncuCyte Caspase 3/7 Green Apoptosis Assay Reagent (Essen BioScience). Four images per well were taken at 2-hour intervals using an IncuCyte ZOOM system (Essen BioScience). The percentage of cell confluency and fluorescent green counts indicating apoptotic cells were measured and analysed by the IncuCyte ZOOM software.

### PDX Experiments

Tumour pieces were implanted subcutaneously in the interscapular fat pad of female NMRI nude, 4-week-old females. Once tumours reached 250 mm³, the mice were enrolled into treatment cohorts. Mice were treated with Tigecycline (50 mg/kg) administered by daily i.p. injection.

No specific randomization method was used. According to animal welfare guidelines, mice were sacrificed at day 18 when the controls reached a critical volume 1200 mm³.

### Example 2. Tigecycline Treatment of Uveal Melanoma Cells

Uveal melanoma cell lines 92.1 (GNAQ mutant with partial deletion of Chromosome 3) and mel0077 (unknown mutational status) where grown in mix 1:1 of F12 and RPMI 1640 and treated with increased concentration of tigecycline (FIG. 1), Chloramphenicol (FIG. 2) or Doxycyline (FIG. 3) Cristal violet staining was performed after 3 days.

### Example 3. Tigecycline in a Mouse Model for Uveal Cancer

For uveal melanoma 2 cohorts (9 mice each) will be treated with a vehicle or with 50 μM Tigecyline administrated daily i.p. Tumour volume is measured over time to identify signs of regression. Progression free survival and overall survival is measured.

Mice will be sacrificed at the human endpoint, i.e. when the tumor will reach 2000 mm³, when the mice will lose more than 20% of their weight or show manifest serious clinical symptoms.

### Example 4. Tigecycline in a Mouse Model for Uveal Cancer

Tigecycline is equally tested in another mouse model as described in EP2925366. Female athymic nude mice (Crl:NU(Ncr)-Foxnlnu, Charles River) of 9 weeks old, and are fed ad libitum.

92.1 uveal melanoma cells are harvested during exponential growth, and resuspended in cold PBS (phosphate buffered saline) with 50% Matrigel™ (BD Biosciences). Each mouse is inoculated subcutaneously in the right flank with 5×10 6 cells (0.2 mL of cell suspension). Tumors were calipered in two dimensions to monitor growth as their mean volume approached the desired 100-150 mm 3 range. Tumor size, in mm³, is calculated from:

Tumor volume=(w²×l)/2

where w=width and l=length, in mm, of the tumor. Tumor weight can be estimated with the assumption that 1 mg is equivalent to 1 mm 3 of tumor volume. Twelve days after tumor cell implantation, animals with individual tumor volumes are measured.

Tigecycline is administered at three times daily (TID) dosing for 21 days, and the reduction of tumor size is determined.

### Example 5. Treatment of Melanoma Cell Lines with Tigecycline

To a uveal melanoma cell line (UM92.1) increasing concentrations of the antibiotic Tigecycline was added. The cells line showed no increase in apoptosis, but a significant and dose-dependent decrease in cell growth (FIG. 4), as measured by activated caspase 3/7 staining and confluency, respectively.

### Example 6. Treatment of Uveal Melanoma PDX Model with Tigecycline

Monotreatment with Tigecycline (50 mg/kg) of the uveal melanoma PDX model Mel-077, derived from a patient progressing on the immune checkpoint inhibitor pembrolizumab (an anti-PD1 agent), was sufficient to stabilise tumour growth and significantly delay progression (FIG. 5). These data indicate that exposure to an antibiotic of the tetracycline family is sufficient to affect the growth of immunotherapy resistant skin and uveal melanomas even when used as a single agent.

### Example 7 Treatment of Uveal Melanoma Cells with Doxycycline

Tigecycline is a relatively expensive antibiotic and it is administered by intravenous infusion, thus increasing discomfort of the patients. Instead, Doxycycline, a broad-spectrum antibiotic belonging to the same family of tetracyclines that exert its anti-bacterial action by binding to the 30S ribosomal subunit to block translation, can be administered orally for extended periods of time with minor adverse effects in patients. Consistently, exposure to increasing concentrations of Doxycycline impaired the growth of melanoma cell cultures exhibiting distinct phenotypic states and carrying different driver mutations in a dose-dependent manner (FIG. 6). In addition, Doxycycline induced in most of the cases a rapid (within 24 to 36 hours) and dose-dependent activation of apoptosis, as measured by caspase activation assay.

1-9. (canceled)

10. A method of treating a uveal melanoma, the method comprising:
administering to a patient with an uveal melanoma an antibiotic that inhibits the 30S ribosomal subunit.

11. The method according to claim 10, wherein the antibiotic is an aminoglycoside or a tetracycline.

12. The method according to claim 11, wherein the tetracycline is a glycylcycline.

13. The method according to claim 12, wherein the glycylcycline is tigecyline.

14. The method according to claim 10, wherein the uveal melanoma is in metastasis.

15. The method according to claim 10, wherein the uveal melanoma carries a mutation in GNAQ, GNA11, BAP1, SF3B1, EIFAX1, or TERT.

16. The method according to claim 10, wherein the antibiotic is administered intravenously, per OS, or intra.

17. The method according to claim 10, wherein the treatment is in combination with another chemotherapy treatment.

18. The method according to claim 10, wherein the treatment is performed in the absence of a radiochemical.
